(19) United States
(12) Patent Application Publication
(10) Pub. No.: US 2009/0024111 A1
(43) Pub. Date: Jan. 22, 2009

(54) Urethral catheter assembly for combining catheterization with injection of therapeutic liquid into the urethral channel

(76) Inventors: German Borodulin, San Francisco, CA (US); Alexander Shkolnik, San Carlos, CA (US)

Correspondence Address: ALEXANDER SHKOLNIK, 485 DARTMOUTH AVENUE, SAN CARLOS, CA, 94070, US

(21) Appl. No.: 11/827,927

(22) Filed: Jul. 16, 2007

(51) Int. Cl. A61M 27/00

(52) U.S. Cl. 604/544

(57) ABSTRACT

A compact ready-to-use urethral catheter assembly suitable for intermittent self-catheterization that comprises a tubular body filled with a therapeutic liquid and having a urinary catheter with a piston portion slidingly installed into the tubular body so that catheterization is accompanied by automatic injection of the therapeutic liquid into the urethra by means of the piston through a gap formed during catheterization between the outer surface of the catheter and the inner walls of the antiseptic cap which is used as a sterile guide for insertion of the catheter without contact with the initial part of the urethra which may be a harbor of infection. The assembly is provided with built-in means for cleaning the urethral meatus before catheterization.

### FIELD OF THE INVENTION

The invention relates to the field of urology, in particular, to a ready-to-use urethral catheter assembly for use in female patients that incorporates means for combining catheterization with introduction into the urethra of lubricating, antiseptic, and anesthetic liquids, individually or in combination, and means for protecting the urethra from introduction of infection during catheterization. The urethral catheter of the invention is convenient for sterile or clean intermittent (short-term) catheterization.

### DESCRIPTION OF PRIOR ART

Intermittent catheterization may be necessary for patients who are unable to completely empty the urinary bladder during urination or unable to naturally urinate by themselves temporarily or permanently. Such a condition is known as urinary retention. Intermittent catheterization may also be required, e.g., for obtaining a sterile urinary specimen from a patient in a doctor's office, or for other reasons.

Intermittent catheterization involves the temporary placement of a catheter through the urethra to the bladder for emptying the latter. A goal of intermittent catheterization is to prevent urinary-tract infection by completely emptying the urinary bladder since accumulation of urine in the bladder over a long period of time may create conditions for development of a urinary-tract infection. After a bladder is effectively drained, urinary-tract infections cease to be a problem, and the kidneys are safeguarded.

It is important that prior to insertion of a urinary catheter, the genital area and the meatus, in particular, must be cleaned, preferably with an antiseptic agent, which, however, is not always available to a patient, especially under self-catheterization conditions. Furthermore, it is known that the first third of the female urethra is a harbor of infection, and therefore there is a risk that insertion of the catheter into the urethra and further into the urinary bladder may introduce an infection into the proximal urethra and bladder.

A device that may partially solve the above problem is a urinary plug for use in female patients described in U.S. Pat. No. 5,806,527 issued in 1998 to G. Borodulin, et al. Although the device is not intended for catheterization but rather for preventing involuntary release of urine from the bladder, the device is equipped with an infection-protective cap fitted on the distal end of the plug's tubular body insertable into the urinary bladder through the urethra. First, the infection-protective plug is inserted into the front infectious portion of the urethra, and then the plug is guided through the infection-protective cap without contact with the infectious part of the urethra.

A similar principle of protection against introduction of infection into the urinary bladder is used in a catheter for use in female patients for self-catheterization described in U.S. Pat. No. 6,544,240 issued in 2003 to Borodulin, et al. The device has a sterile infection-protective cap slidingly fitted onto the distal end of said catheter. The cap has a slit on its distal end for possibility of pushing the catheter through the sterile tip into the bladder without physical contact of the catheter with the infectious front part of the urethra.

However, the devices of U.S. Pat. No. 5,806,527 and U.S. Pat. No. 6,544,240 do not have any means that would pre-treat the urethra or facilitate introduction of the plug or catheter into the bladder by treating the urethra during catheterization, e.g., by lubricating the urethral channel, etc.

Urinary catheters supplied for intermittent catheterization in general must have a lubricant applied to the outer surfaces thereof to facilitate insertion into the urethra. For example, hydrophilic urinary catheters have a hydrophilic outer surface coating that should be wetted with fluid such as water or a saline solution for a certain time period before insertion thereof into the urethra of a patient for lubrication purposes.

Various methods for lubricating urinary catheters have been previously proposed, examples of which are given below.

U.S. Pat. No. 5,209,726 makes known a self-lubricating urinary catheter and a method for self-catheterization. The catheter has an annular lubricant reservoir surrounding an inner tube having perforations. As the catheter is inserted into the urethra of a patient, the lubricant is forced from the reservoir into the inner tube and out into the urethra through a discharge outlet in the catheter. Lubrication of the outer surface of the catheter, therefore, only takes place as the catheter is being inserted into the urethra of the patient, resulting in at least some initial discomfort for the patient on insertion of the catheter.

U.S. Pat. No. 3,967,728 makes known a catheter package comprising a urinary catheter and a rupturable, lubricant-containing pouch. One edge of the pouch is located within the package adjacent to the tip of the catheter, and the seal at that edge is constructed so that it ruptures when the pouch is squeezed for lubricating the tip of the catheter. Because only the tip of the catheter is lubricated, the patient still suffers discomfort on insertion of the catheter into the urethra.

U.S. Pat. No. 7.066,912 issued in 2006 to Nestenborg, et al., describes a wetting apparatus for wetting a hydrophilic urinary catheter comprising a wetting-fluid container that holds a wetting fluid and that can be opened by application of a pulling force thereto; a wetting receptacle; and a hydrophilic urinary catheter to be wetted by said wetting fluid and being arranged within said wetting receptacle. The wetting-fluid container is arranged within the wetting receptacle, and the wetting receptacle is extendable for opening the wetting container without rupturing the sealed condition of the wetting receptacle.

Another example of a catheter suitable for self-catheterization is a compact urinary prepacked catheter developed by Coloplast Company (Denmark) and known under trademark SpeediCath™ that can be obtained in a sterile and ready-to-use prehydrated form right out of the package. Each SpeediCath™ comes prepacked in sterile saline solution and in a container that can be taken anywhere, for example, similar to a ballpoint pen. Since SpeediCath™ is prelubricated, its coating is always optimally hydrated to be slippery and therefore comfortable.

Although the SpeediCath™ catheter described above provides a catheter in a ready-to-use form and in a sterile state, it does not guarantee prevention of infection that can be introduced into the urethra during insertion unless the urethral meatus has been thoroughly cleaned before catheterization. Unfortunately, means for cleaning the meatus are not always readily available under conditions where self-catheterization is performed.

However, neither the SpeediCath™ catheter nor any other catheter mentioned above or known to the applicants possesses self-contained and readily available means that simultaneously combine features such as protection against introduction of infection and provision for cleaning the urethral meatus and treating the urethra, e.g., by automatically introducing a lubricant, an anesthetic medium, or an antiseptic substance, or combination thereof, during the process of catheterization.

### OBJECTS AND SUMMARY OF THE INVENTION

It is an object of the present invention to provide a catheter assembly for use in female patients that has means for combining catheterization with introduction into the urethra of lubricating, antiseptic, or anesthetic liquids, individually or in combination. It is another object to provide the aforementioned catheter assembly in a self-contained and ready-to-use form convenient for self-catheterization. A further object is to provide the aforementioned catheter assembly with a sterile anti-infectious cap connected to the liquid-containing tubular body of the catheter assembly. A further object is to provide the sterile anti-infectious cap with a protective cover that is reliably attached to the cap and protects the cap from contamination. Another object is to provide the aforementioned catheter with means for cleaning the entrance into the urethra before catheterization.

The device of the invention is a catheter assembly for use in female patients that comprises essentially a urinary catheter that passes through a hollow tubular body filled with a urethra-treating liquid, e.g., a lubricating liquid, anesthetic liquid, antiseptic liquid, physiological solution, or any combination of the above. The proximal end of the tubular body is closed with the piston of a plunger, while the distal end of the tubular body is closed with an anti-infectious cap that is connected to the tubular body. The piston is temporary and releasably fixed to the end face of the tubular body with the possibility of pushing it into the interior of the tubular body by manual force, and the anti-infectious cap has a slit through which the distal end of the catheter can pass and protrude farther out from the device when the piston is shifted forward inside the tubular body. In order to seal the interior of the tubular body during storage of the catheter assembly before catheter use, the catheter has a portion of an increased diameter on the distal end that in a non-operative position is tightly inserted into the interior of the anti-infectious plug and prevents leakage of the liquid through the plug's slit. The catheter inlet opening that is intended for release of urine from the urinary bladder through the catheter, e.g., to the urine-collecting bag, is formed in the aforementioned increased-diameter portion and is overlapped by the inner walls of the anti-infectious cap. The opposite side of the tubular body is closed by the aforementioned piston. The anti-infectious cap is covered with a protective cover that is threaded onto the external thread of the tubular body to protect the anti-infectious cap from contamination.

In use, first the protective cover is disconnected, the anti-infectious cap is inserted into the front infectious part of the urethra until further movement thereof is impossible, and then the plunger is pushed forward and is moved toward the urethra. As soon as the enlarged-diameter portion on the distal end of the catheter passes into the urethra through the slit of the anti-infectious cap, an annular gap is formed between the inner walls of the anti-infectious cap and the outer surface of the small-diameter portion of the catheter. As a result, movement of the piston in the forward direction pumps the liquid from the interior of the tubular body and farther through the aforementioned annular gap and the anti-infectious cap into the urethra. If the liquid is a lubricant, it will facilitate the insertion of the catheter into the urethra. If the liquid is an antiseptic solution, it will perform antiseptic treatment of the urethra, and if the liquid is an anesthetic solution, it will provide painless insertion of the catheter and subsequent post-catheterization treatment. The liquid may comprise a mixture of two or all three components in appropriate proportions. The catheter is molded from a soft biologically acceptable plastic and is disposable.

### BRIEF DESCRIPTION OF THE DRAWINGS

FIG. 1 is a longitudinal sectional view of a catheter assembly of the invention in the sealed nonoperative condition in which it is stored.

FIG. 2 is a longitudinal sectional view of the catheter assembly of FIG. 1 in the operative condition when it is inserted into the urethra.

FIG. 3 is a general view of a modified catheter assembly of the invention with means for cleaning the urethral meatus and the surrounding area.

FIG. 4 is a fragmental sectional view of the catheter of FIG. 3 illustrating a position of a pre-impregnated compressed pad that can be used for cleaning the urethra and the surrounding areas before catheterization.

FIG. 5 is a sectional view of the cap of FIG. 4 with the compressed pad in an expanded state and ready for use.

FIG. 6 is view similar to FIG. 4 but for another modification of the cap with a compressed sanitary pad.

FIG. 7 is a view similar to FIG. 6 but with the catheter shifted forward for forming a gap through which the liquid contained in the tubular body of the assembly may leak to the sponged body.

FIG. 8 shows the impregnated sponged body of the device of FIG. 7 in the expanded state and ready for use.

### DETAILED DESCRIPTION OF THE INVENTION

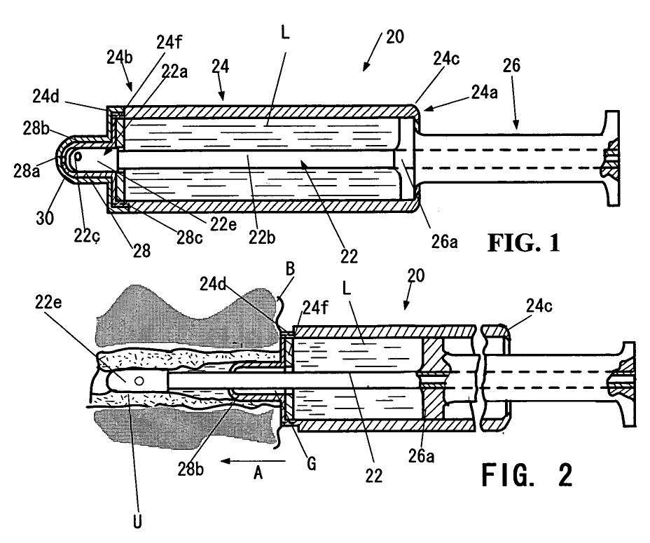

A catheter assembly of the invention for use in female patients, which as a whole is designated by reference numeral 20, is shown in FIGS. 1 and 2, where FIG. 1 is a longitudinal sectional view of a catheter assembly 20 in the sealed nonoperative condition in which it is stored, and FIG. 2 is a longitudinal sectional view of the catheter assembly 20 in the operative condition when it is inserted into the urethra U.

The catheter assembly 20 comprises essentially a urinary catheter 22 that passes through a hollow tubular body 24 filled with a urethra-treating liquid L. This liquid may comprise, e.g., a lubricating medium, anesthetic liquid, antiseptic liquid, physiological solution, or any combination of the above.

The proximal end 24a of the tubular body 24 is closed with a piston 26a of a plunger 26, while the distal end 24b of the tubular body 24 is closed with an anti-infectious cap 28 that is rigidly attached to the distal end of the hollow tubular body 24. The cap 28 has a protruding cylindrical portion 28b with a rounded tip and a flat flanged portion 28c. Reference numeral 24f designates the front end face or bottom portion of the tubular body 24 to which the flanged portion 28c can be attached, e.g., by gluing or by thermal welding.

The piston 26a is temporary and releasably fixed to the proximal end face 24c of the tubular body 24, e.g., by press fit, with the possibility of pushing it into the interior of the tubular body by manual force, and the anti-infectious cap 28 has a slit 28a on the rounded tip of the protruding portion 28b through which the distal end 22a of the catheter 22 can pass and protrude farther out from the device 20 when the piston 26a is shifted forward inside the tubular body 24 in the direction of arrow A in FIG. 2.

In order to seal the interior of the tubular body 24 during storage of the catheter assembly 20 before catheter assembly use, the catheter 22 has a small-diameter portion 22b, which corresponds essentially to the major part of its length, and a portion 22e of an increased diameter on its the distal end 22a that in a nonoperative or storage position is tightly inserted into the interior of the anti-infectious plug 28 and prevents leakage of the liquid L through the plug's slit 28a.

The catheter inlet opening 22c that is intended for release of the urine from the urinary bladder (not shown) through the catheter 22, e.g., to a urine-collecting bag (not shown), is formed in the aforementioned increased-diameter portion 22e and in the inoperative condition is overlapped by the inner walls of the anti-infectious plug 28. The opposite side, e.g., the proximal 24a of the tubular body 24 is closed by the aforementioned piston 26a. The anti-infectious plug 28 is covered with a protective cover 30 that is threaded onto the external thread 24d of the tubular body 24 to protect the anti-infectious plug 28 from contamination.

In use, first the protective cover 30 (FIG. 1) is disconnected from the catheter assembly 20 by untwisting from the thread 24d, the entrance into the urethra is cleaned (if such cleaning means are available), the anti-infectious plug 28 is inserted into the front infectious part of the urethra U until contact of the flange portion 28c of the anti-infectious cap with the patient's body B (FIG. 2). Contact with the body seals the urethra and provides guiding of the liquid L directly into the urethra U without leakage to the outside.

Following this, the catheter 22 is shifted forward in the direction of arrow A in FIG. 2.

As soon as the enlarged-diameter portion 22e on the distal end of the catheter 22 passes into the urethra U through the slit 28a of the anti-infectious cap 28, an annular gap G (FIG. 2) is formed between the inner walls of the anti-infectious cap 28 and the outer surface of the small-diameter portion 22b of the catheter 22. As a result, movement of the piston 26a in the forward direction pumps the liquid L from the interior of the tubular body 24 and farther through the aforementioned annular gap G in the anti-infectious cap 28 into the urethra U. If the liquid L is a lubricant, it will facilitate insertion of the catheter 22 into the urethra U. If the liquid L is an antiseptic solution, it will perform antiseptic treatment of the urethra U, and if the liquid L is an anesthetic solution, it will provide painless insertion of the catheter and subsequent post-catheterization treatment. The liquid L may comprise a mixture of two or all three components in appropriate proportions. The catheter is molded from a soft biologically acceptable plastic and is disposable.

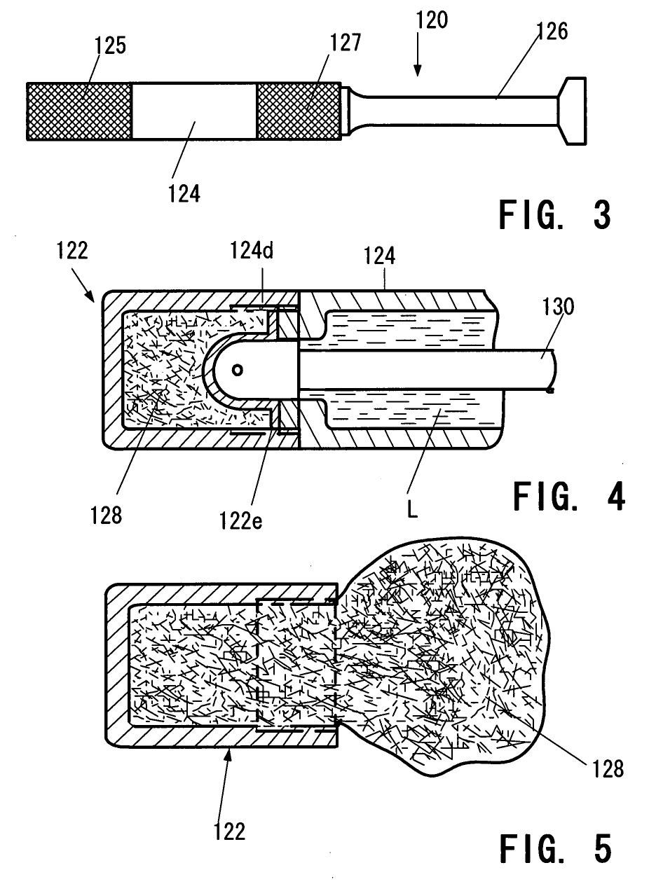

FIG. 3 is a general view of a urinary catheter assembly 120 with built-in means for cleaning the urethral meatus and the adjacent area before self-catheterization. FIG. 4 is a fragmental sectional view of the distal end of the catheter assembly of FIG. 3, and FIG. 5 shows means for cleaning the urethra meatus before catheterization in a ready-to-use condition.

Since, in general, the device of FIG. 3 is the same as described in the previous embodiment, only those parts thereof that are different will be described in the specification and shown in the drawings. The catheter assembly of FIGS. 3 to 5 differs from that shown in FIGS. 1 and 2 by provision of a cap 122 that is screwed onto the thread 124d formed on the distal end of the tubular body 124 of the catheter assembly. However, as shown in FIG. 4, the interior of the cap 122 is filled with an impregnatable and compressible resilient material such as a compressed sponged pad 128. This pad 128 is pre-impregnated with an antiseptic solution. When the urinary catheter assembly is in a stored state, as shown in FIGS. 3 and 4, the cap 122 is closed, and the interior thereof with the pre-impregnated pad 128 is sealed by an increased-diameter portion 122e of the catheter 130.

As shown in the embodiments of FIGS. 1 and 2, the interior of the tubular body 124 is filled with the antiseptic or anesthetic liquid L (FIG. 3), which can be injected into the urethra by pushing the plunger 126 in the forward direction, as in the previous embodiment.

Reference numerals 125 and 127 designate knurled portions on the surface of the cap 122 and the tubular body 124, respectively, which facilitate untwisting of the cap 122 and holding of the tubular body 124 during pushing of the plunger. When prior to use of the urinary catheter assembly 120 the cap is untwisted from the thread 124d and is disconnected from the tubular body 124, the released compressed sponged pad 128 expands and protrudes out from the interior of the cap 122 in the form shown in FIG. 5. Since the sponged pad 128 is pre-impregnated with an antiseptic solution, it can be used for cleaning the urethral meatus and the surrounding area, thus providing an additional sanitation means for protecting the urethra from introduction of infective matter. After use, the cap 122 and the sponged body 128 are discarded.

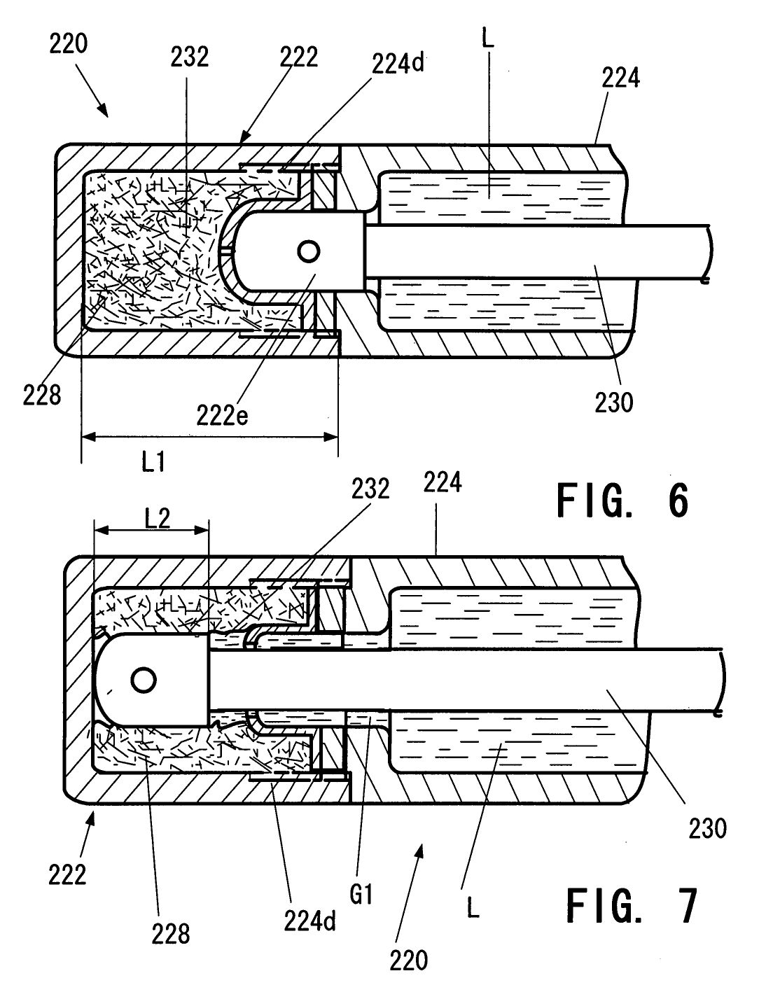

FIGS. 6, 7, and 8 illustrate another modification of a catheter assembly 220 with the pre-impregnated sponged pad. Since, in general, the catheter assembly 220 of FIGS. 6, 7, and 8 is the same as that described in the previous embodiments, only those parts thereof that are different will be described in the specification and shown in the drawings.

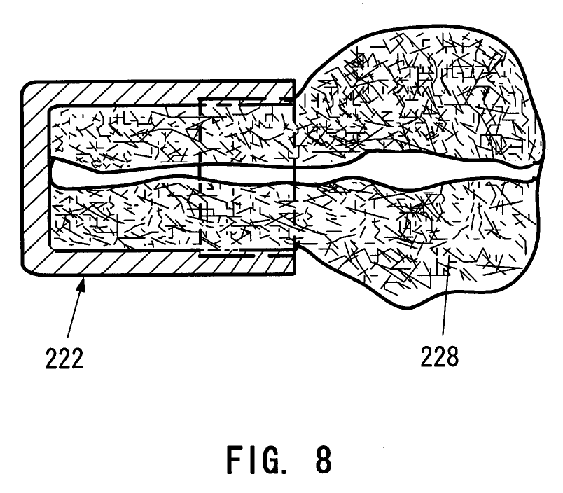

Similar to the modification shown in FIGS. 3 to 5, the catheter assembly 220 has a cap 222 screwed onto a thread 224d formed on the distal end of a tubular body 224. The modification shown in FIGS. 6 to 8 differs from that shown in FIGS. 3 and 5 in that the compressed sponged pad may not be pre-impregnated with an antiseptic liquid but can be soaked with the same liquid L that is stored in the tubular body 224. For this purpose, the interior of the sponged pad 228 has a central recess 232 (FIG. 6) the length L1 of which (FIG. 7) is greater than the length L2 of the increased-diameter portion 222e of the catheter so that there is sufficient space for pushing the increased-diameter portion 222e of the catheter 230 into the cap 222 in order to form a gap G1 prior to use of the device. This condition is shown in FIG. 7. The liquid L stored in the tubular body 224 flows through this gap G1 into the interior of the cap 222 and impregnates the compressed sponged pad 228 before use thereof for cleaning the urethral meatus. In the state of storage of the catheter assembly 220, i.e., before use of the plunger (not shown in FIGS. 6 and 7), the interior of the cap 222 is sealed from penetration of the liquid L. FIG. 8 shows the impregnated sponged pad 228 in the expanded state and ready for use.

Thus, it has been shown that the present invention provides a catheter assembly for use in female patients that has means for combining catheterization with introduction into the urethra of lubricating, antiseptic, anesthetic liquids, individually or in combination. The catheter assembly is in a self-contained and ready-to-use form convenient for assisted or self-catheterization. The catheter assembly is provided with a sterile anti-infectious cap made integrally with the body of the catheter. The sterile anti-infectious cap is provided with a protective cover that is reliably attached to the cap and protects it against contamination. Finally, the catheter assembly of the invention is provided with built-in means for cleaning the entrance into the urethra before catheterization.

Although the invention has been shown and described with reference to specific embodiments, these embodiments should not be construed as limiting the areas of application of the invention and that any changes and modifications are possible provided these changes and modifications do not depart from the scope of the attached patent claims. For example, the catheter assembly of the invention is not used only for self-catheterization but may also be used for catheterization of patients by medical personnel in hospitals or outpatient clinics. The catheter can be used for introduction of any therapeutic liquid into the urethra and into any other duct. The plunger, piston, and catheter can be molded as a single piece. Although the urethral catheter of the invention is described as a catheter for use in female patients, with some minor changes in geometry, this catheter can be used for male patients.

1. A urethral catheter assembly for urethral catheterization comprising:
a hollow casing that has a distal end, a bottom portion with an opening at said distal end, a proximal end, and an interior;
a catheter moveably installed in the hollow casing and having a distal end and a proximal end;
an anti-infectious cap attached to the distal end of the hollow casing and intended for guiding the catheter into the urethra without contact with the urethral wall in the vicinity of the urethral meatus, said anti-infective cap having a slit for passing the catheter;
a therapeutic liquid that fills the interior of the hollow casing; and
a means for injecting therapeutic liquid into the urethra simultaneously with urethral catheterization; the catheter assembly having a storage condition in which the catheter is stored inside the hollow casing and a catheterization condition in which the catheter projects from the distal end of the hollow casing.

2. The urethral catheter assembly of claim 1, wherein the catheter has a distal end insertable into the urethra and a proximal end on the side opposite to the distal end, the aforementioned means for injection of therapeutic liquid into the urethra simultaneously with catheterization comprising a piston that is slidingly and sealingly installed in the interior of the hollow casing and rigidly connected to the catheter, the distal end of the catheter passing through the aforementioned piston and through the aforementioned opening of said bottom portion to the outside of the hollow casing; in said storage condition of the catheter assembly, the aforementioned therapeutic liquid being sealed in the hollow casing between the aforementioned anti-infective cap and the piston.

3. The urethral catheter assembly of claim 2, further provided with means for sealing therapeutic liquid in said storage condition of the catheter assembly.

4. The urethral catheter assembly of claim 3, wherein the means for sealing comprises the aforementioned piston and a portion of an increased diameter on the distal end of the catheter that is sealingly inserted through said opening in the bottom portion into the anti-infective cap, said catheter having a portion of reduced diameter that is smaller than the portion of the increased diameter and that extends from the portion of the increased diameter to the piston.

5. The urethral catheter assembly of claim 1, further provided with a removable protective cover for covering the aforementioned anti-infective cap and for protecting thereof from contamination.

6. The urethral catheter assembly of claim 2, further provided with a removable protective cover for covering the aforementioned anti-infective cap and for protecting thereof from contamination.

7. The urethral catheter assembly of claim 4, further provided with a removable protective cover for covering the aforementioned anti-infective cap and for protecting thereof from contamination.

8. The urethral catheter assembly of claim 1, wherein the proximal end of the catheter that passes through the piston to the outside of the proximal end of the hollow casing is made in the form of a pusher for slidingly pushing the piston into the hollow casing and thus for injecting therapeutic liquid into the urethra.

9. The urethral catheter assembly of claim 3, wherein the proximal end of the catheter that passes through the piston to the outside of the proximal end of the hollow casing is made in the form of a pusher for slidingly pushing the piston into the hollow casing and thus for injecting therapeutic liquid into the urethra.

10. The urethral catheter assembly of claim 4, wherein the proximal end of the catheter that passes through the piston to the outside of the proximal end of the hollow casing is made in the form of a pusher for slidingly pushing the piston into the hollow casing and thus for injecting therapeutic liquid into the urethra.

11. The urethral catheter assembly of claim 7, wherein the proximal end of the catheter that passes through the piston to the outside of the proximal end of the hollow casing is made in the form of a pusher for slidingly pushing the piston into the hollow casing and thus for injecting therapeutic liquid into the urethra.

12. The urethral catheter assembly of claim 5, further provided with an impregnatable, compressible, and resilient body that in said storage condition of the catheter assembly is stored in the protective cap in a compressed state.

13. The urethral catheter assembly of claim 12, wherein the means for sealing comprises the aforementioned piston and a portion of an increased diameter on the distal end of the catheter that is sealingly inserted into the anti-infective cap, said catheter having a portion of a reduced diameter that is smaller than the diameter of the aforementioned portion of an increased diameter and that extends from the portion of the increased diameter to the piston.

14. The urethral catheter assembly claim 12, wherein the impregnatable, compressible, and resilient body is preimpregnated with a therapeutic liquid.

15. The urethral catheter assembly of claim 13, wherein the protective cap has a cavity that contains said impregnatable, compressible, and resilient body, said cavity having a first length; the aforementioned portion of an increased diameter on the distal end of the catheter has a second length that is shorter than said first length so that a gap for passing therapeutic liquid to the impregnatable, compressible, and resilient body is formed between the opening of the bottom portion and the portion of the reduced diameter for impregnating said impregnatable, compressible, and resilient body when the portion of the increased diameter is shifted toward said bottom portion into the impregnatable, compressible, and resilient body.

16. The urethral catheter of claim 15, wherein the impregnatable, compressible, and resilient body has an axial opening for facilitating insertion of the increased-diameter portion into the impregnatable, compressible, and resilient body when this body is in the aforementioned compressed state.

17. The urethral catheter of claim 16, wherein the therapeutic liquid is selected from lubricating liquid, anesthetic liquid, or antiseptic liquid.

18. The urethral catheter of claim 12, wherein the therapeutic liquid is selected from lubricating liquid, anesthetic liquid, or antiseptic liquid.

19. A method of urethral catheterization by means of a catheter assembly simultaneously with introduction into the urethra of a therapeutic liquid, said method:
providing a catheter assembly in the form of a tubular body filled with a therapeutic liquid and having a urinary catheter with a piston portion slidingly installed in said tubular body;
providing the tubular body with an anti-infectious cap;
inserting the anti-infectious cap into the patient's urethra; and
performing catheterization by inserting the catheter into the urethra through said anti-infections cap and simultaneously injecting therapeutic liquid into the urethra through said anti-infectious cap by displacing the therapeutic liquid from the tubular body by means of the piston portion of the catheter.

20. The method of urethral catheterization according to claim 19, further:
providing the catheter assembly with a protective cap that covers the aforementioned anti-infectious cap with an impregnatable, compressible, and resilient body, which is preimpregnated with an antiseptic liquid;
compressing the impregnatable, compressible, and resilient body for storing in the protective cap and attaching the protective cap to the catheter assembly;
disconnecting the protective cap from the catheter assembly before catheterization, thus allowing the impregnatable, compressible, and resilient body to resiliently expand and project beyond the protective cap;
carrying out antiseptic cleaning of the entrance into the urethra and of the surrounding area with the use of said impregnatable, compressible, and resilient body which has been preimpregnated with the antiseptic liquid; and then
carrying out the aforementioned steps of inserting the anti-infectious cap into the patient's urethra and performing catheterization by inserting the catheter into the urethra through said anti-infections cap and simultaneously injecting the therapeutic liquid into the urethra through said anti-infectious cap by displacing the therapeutic liquid from the tubular body by means of the piston portion of the catheter.
